(19) United States
(12) Patent Application Publication
(10) Pub. No.: US 2016/0059031 A1
(43) Pub. Date: Mar. 3, 2016

(54) Catheter/Stent System For Activation of Photodynamic Therapy Within The Catheter/Stent System

(71) Applicant: University of Rochester, Rochester, NY (US)

(72) Inventors: Andrew Wescott, Rochester, NY (US); Yvonne Teng, Henderson, NV (US); Ting Zheng, Rochester, NY (US); Xuxin Zhang, Brooklyn, NY (US); Thomas H. Foster, Rochester, NY (US)

(21) Appl. No.: 14/410,102

(22) PCT Filed: Jun. 26, 2013
(86) PCT No.: PCT/US13/47906

§ 371 Date: Dec. 21, 2014

Related U.S. Application Data
(60) Provisional application No. 61/664,496, filed on Jun. 26, 2012.

(51) Int. Cl. A61N 5/06

(52) U.S. Cl. CPC A61N 5/062; A61N 5/0601; A61N 5/0624; A61N 2005/0652

(57) ABSTRACT

A catheter/stent has multiple LEDs set in it along its length, connected to a voltage source such as a medical battery. The catheter/stent is inserted into the bile duct, to provide relief for tumor in growth that would otherwise obstruct the flow of bile through the bile duct. The LEDs perform photodynamic therapy to keep the lumen of the catheter free of tumor cells and bacteria.

### REFERENCE TO RELATED APPLICATIONS

The present application claims the benefit of U.S. Provisional Patent Application No. 61/664496 filed Jun. 26, 2011, and to International Application No. PCT/US13/47906 filed Jun. 26, 2013, whose disclosures are incorporated herein by reference in their entirety.

### FIELD OF THE INVENTION

The present invention is directed to catheters and stems and more particularly to catheters and stems capable of providing photodynamic therapy to prevent the growth of cancer tissue or bacteria within the catheters and stents.

### DESCRIPTION OF RELATED ART

Bile is a liquid released by the liver that aids in fat digestion in the body. Bile is secreted by the liver and travels through the bile ducts to the gall bladder and small intestine. Tumor and bacterial in-growth within the bile duct obstructs the flow of bile and causes bile build up within the liver, which results in painful inflammation and infection.

Cholangiocarcinoma is a form of cancer in which tumors grow within the bile duct. Cholangiocarcinoma typically is not diagnosed until advanced stages, when the bile duct is already obstructed by tumor cells. The tumor is often unresectable, and only palliative treatment can be administered. Each year, approximately 2,000 to 3,000 new cases are diagnosed in the United States, translating into an annual incidence of 1-2 cases per 100,000 people. The prognosis of cholangiocarcinoma is poor, with survival rates of less than 5% over a 5-year span.

A common palliative treatment is biliary stenting, where a biliary catheter is inserted percutaneously into the bile ducts to relieve occlusion and allow drainage of bile fluid. Unfortunately, overgrowth of tumor cells and/or bacteria within the stent occurs over time to occlude the lumen of the catheter/stent, leading to infections and bile fluid buildup. Consequently, these devices need to be replaced periodically—approximately every six to eight weeks. While biliary stenting provides relief for the patient, the median survival time for cholangiocarcinoma patients who undergo biliary stenting alone is short and quality of life is challenging.

Another treatment option is photodynamic therapy (PDT). Currently approved PDT drugs such as Photofrin have been used to treat cholangiocarcinoma at the time of initial stent placement.

A similar application of photodynamic therapy is found in the use of PDT within endotracheal tubes to eradicate accumulated biofilm. The method, disclosed in U.S. Patent Application Publication No. US 2002/0091424 A1 to Biel, utilizes an external light source coupled with an inserted optical fiber catheter to illuminate an endotracheal tube sprayed with photosensitizer to eradicate bacteria. Biel conducted an experiment using photodynamic therapy within endotracheal tubes and concluded that photodynamic therapy greatly reduces biofilm accumulation. Unfortunately, that method is not feasible for biliary stenting, as a biliary catheter is much smaller in diameter than an endotracheal tube. Insertion of the optical fiber is invasive and will prevent proper bile drainage; thus, such insertion cannot be a longterm solution for the bile duct.

Another similar technique is disclosed in U.S. Pat. No. 6,551,346 B2 to Crossley. The patent discloses a method of applying of photodynamic therapy to prevent infection within catheter lumens; the catheter type is unspecified. Photodynamic therapy is activated through multiple optical fiber diffusers within the catheter wall that are illuminated through an external light source. No products seem to be commercially available, likely due to the difficulty in the manufacturing process and expenses.

### SUMMARY OF THE INVENTION

A need thus exists in the art to provide a practical technique for biliary stenting and maintenance of stent patency that overcomes the above difficulties. It is therefore an object of the invention to provide a practical biliary stent/catheter system with the ability to remove occlusion via activation of photodynamic therapy within the device lumen to eliminate the need for frequent stent/catheter replacement.

To achieve the above and other objects, the present invention is directed to a biliary catheter system with the ability to administer antimicrobial/anti-tumor photodynamic therapy to eradicate tumor cells and bacteria, thereby reducing the chance of biliary obstruction and infection. Photodynamic therapy is chosen as the method because it is noninvasive, nontoxic, and already in use to treat cholangiocarcinoma, While the present invention, is focused on biliary catheters/stents, and the potential market is directed towards the gastroenterology and interventional radiology communities, the inherent technology, once developed, can be applied to various other catheters/stents and in other areas of medicine.

The stent/catheter system includes a system of embedded LEDs, for illuminating the internal lumen of a biliary stent or catheter. The aim of the device is to utilize photodynamic therapy (PDT) to eradicate bile duct cancer cell overgrowth and/or microbial colonization within the catheter/stent lumen.

The device is inserted, by the physician percutaneously into the common bile duct to provide a path for bile to flow. As necessary, the patient comes to the hospital for an injection of photosensitizer drug from the physician, and the device is activated for sterilization of the stent.

### BRIEF DESCRIPTION OF THE DRAWINGS

A preferred embodiment of the present invention will be set forth in detail with reference to the drawings, in which:

FIG. 1 is a schematic diagram showing a catheter/stent device according to the preferred embodiment;

FIG. 2A is a circuit diagram showing a parallel wiring arrangement usable in the device of FIG. 1; and

FIG. 2B is a circuit diagram showing a series wiring arrangement usable in the device of FIG. 1.

### DETAILED DESCRIPTION OF THE PREFERRED EMBODIMENT

A preferred embodiment of the present invention will be set forth in detail with reference to the drawings, in which like reference numerals refer to like elements or steps throughout.

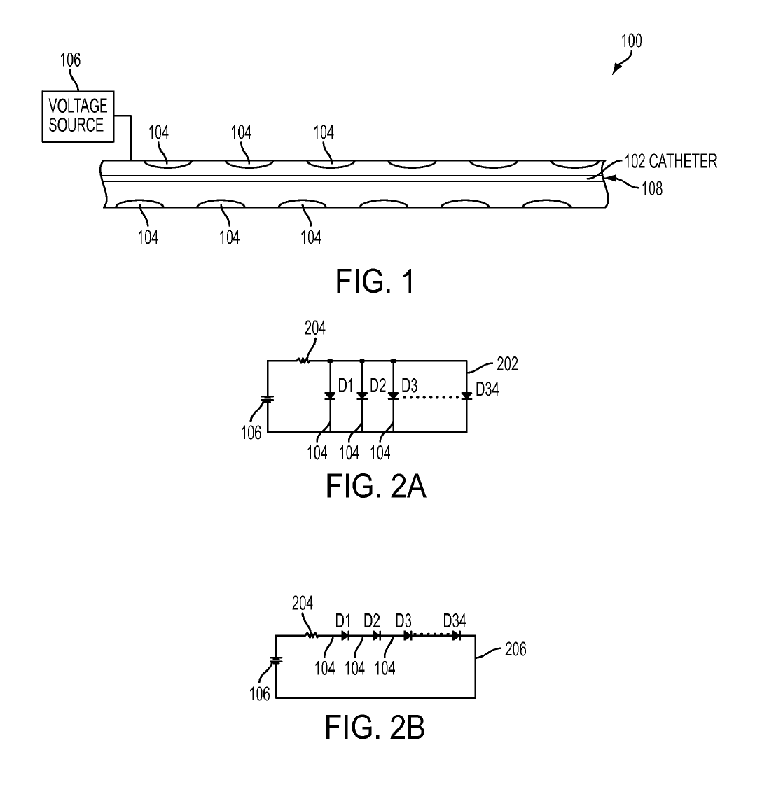

FIG. 1 is a schematic diagram showing a catheter/stent device 100 according to the preferred embodiment. A catheter 102 has multiple LEDs 104 set in it along its length, connected to a voltage source 106 such as a medical battery. The voltage source 106 can be located inside or outside of the patient's body, although it preferably has some way for the physician to turn it on and off for PDT. The LEDs perform PDT to keep the lumen 108 of the catheter 102 free of tumor cells and bacteria.

The catheter and the LEDs in one embodiment preferably have the following specifications:

Functional

Power output within lumen: >10 MW CM-2

Output wavelength: typically deep red, photosensitizer specific

Performance

Heat generation: temperature increase <1° C.

Biocompatible

Sterilizable

Interface

External diameter: 12 Fr±4 Fr

Internal diameter: 1.3 mm±0.05 mm

Length: variable

The device can be utilized by either an interventional radiologist or gastroenterologist. As such, the device will need to be able to be advanced to the bile duct similarly to existing biliary catheter/stent devices. The catheter/stent will also be placed within the patient for an extended period of time. One material with these properties is polyethylene. Future devices can he made with the ability to be implanted via endoscopy (such as Advanix) or through percutaneous catheter insertion.

Issues with embedding LEDs within the catheter tube are the issues of biocompatibility and heat generation. This has been assessed in the selection of lead-free LEDs as well as the use of biocompatible cement and silicone as an insulator. Testing of the heat generation was conducted; preliminary results showed no increase in temperature with LED activation for 30 minutes.

In order to activate the photosensitizer, we generate red: light (630-780 nm) at an intensity profile of 10-100 mW/cm2.

Commercially available LEDs emit light at different wavelengths. They are available in different sizes. A typical LED is powered by a constant current, The LEDs selected for our preliminary design are LS Q976 LEDs manufactured by Osram. The forward current and voltage for each LED is 20 mA and 2 V, respectively.

Two circuit designs are proposed. The circuit 202 of FIG. 2A connects the LEDs 104 in parallel to the voltage source 106 and at least one stabilizing resistor 204, while the circuit 20 of FIG. 2B connects the LEDs 104 in series. Each of the designs has its own advantages and disadvantages.

Connecting the LEDs in parallel has distinct advantages over connection in series. A parallel circuit delivers a constant voltage drop across each LED semi-conductor and enables use of a low voltage source to power all of the LEDs simultaneously. However, although the parallel connection ensures a constant voltage drop across each LED in the circuit, it cannot ensure an equal current through each LED. If one of the LEDs were poorly manufactured so that its apparent resistance is larger than that of the rest of the LEDs, the other LEDs in this circuit would encounter a significantly larger current.

Based on such concerns, there may be advantages to connecting the LEDs in series. The possible limitation in this case is the need for higher voltages.

We elected to create a 12-LED train connected in series with a 330 resistor to stabilize the current. LED intensity varied linearly with input voltage, which is to be expected. The input voltage range for illuminating our LED train was 20-28 V; at 28 V, our device reached a 20 mA current that is the threshold current beyond which these LEDs risked failure.

LEDs are deemed relatively safe in terms of radiation as they are considered different from laser diodes and are not subject to the Federal laser product performance standard. The specific chip surface-mount LED LS Q976 Super-red by Osram was chosen based on its suitable wavelength and appropriate size to fit within our catheter wall without compromising the catheter lumen for drainage.

Features if that LED:

Smallest package 1.6 mm×0.8 mm×0.8 mm

Wavelength: 633 nm (super-red)

Viewing angle: extremely wide (160°)

Optical efficiency: 71 m/W (super-red)

For large-scale production, it is likely that a specially designed polymer mold would be designed to allow for direct incorporation of the LEDs and wires via extrusion.

While both CNC milling and thermo-puncture are viable options, there are challenges associated with applying these techniques to a polymer tube. Research conducted in drainage catheter hole generation revealed catheter hole punch machines. Companies such as Die Technology Inc. specialize in catheter hole punch techniques and have the capacity to punch holes as small as 0.3048 mm in diameter. This would be ideal for creating precisely located and clean tube holes within our device. For ease of manufacturing, it may be advantageous to switch to a circular LED or to design a system with a smaller circular casing or no casing at all.

The input voltage needed to maximally power our train of LEDs connected in series is 28 V. Ideally, the input voltage would be near 6 V, which could more easily be generated from small lithium medical grade batteries. This could likely be achieved via a parallel connection design with incorporation of individual resistors for each LED.

The ability to machine print or manufacture our circuit on a stable platform would he highly advantageous. One of our main concerns with future designs is to create a well connected, stable, and secure circuit that is reliable. The use of a flexible circuit board would allow us to maintain tube flexibility while precisely positioning LEDs with respect to one another and to holes within the tube. In the event of parallel connection, the resistor size and position would have to be taken into account for embedding. There is also the potential to use a very thin walled stent or catheter tube (similar to the Advanix system) and simply place the board inside the tube with an adhesive.

As another option, commercially available technology enables application of flowable materials to substrates, which include polymers and cylindrical surfaces.

While a preferred embodiment has been set forth in detail above, those skilled in the art who have reviewed the present disclosure will readily appreciate that other embodiments can be realized within the scope of the invention. For example, numerical values are illustrative rather than limiting, as are recitations of particular materials and particular sources of components. Therefore, the present invention should be construed as limited only by the appended claims.

What is claimed is:

1. A system for biliary stenting, the system comprising:
a biliary stent or catheter;
a plurality of light-emitting diodes in a wall of the biliary stent or catheter for emitting light into a lumen of the biliary stent or catheter; and
electrical connections for connecting the plurality of light-emitting diodes to a power source.

2. The system of claim 1, wherein the electrical connections connect the plurality of light emitting diodes in series.

3. The system of claim 1, wherein the electrical connections connect the plurality of light emitting diodes in parallel.

4. The system of claim 1, wherein the electrical connections connect the plurality of light emitting diodes using hybrid parallel and series circuitry.

5. The system of claim 1, wherein the biliary stent or catheter has an external diameter in a range of 12 Fr±4 Fr.

6. The system of claim 1, wherein the biliary stent or catheter has an internal diameter in a range of 1.3 mm±0.05 mm.

7. A method for biliary stenting in a bile duct in a patient, the method comprising;
a) inserting a biliary stent or catheter into the bile duct, the biliary stent or catheter having in a plurality of light-emitting diodes disposed in a wall of the biliary stent or catheter for emitting light into a lumen of the biliary stent or catheter and electrical connections for connecting the plurality of light-emitting diodes to a power source;
b) supplying a photosensitizer to the patient; and
c) supplying power from the power source to the plurality of light-emitting diodes to excite the photosensitizer for photodynamic therapy.

8. The method of claim 7, wherein the electrical connections connect the plurality of light-emitting diodes in series.

9. The method of claim 7, wherein the electrical connections connect the plurality of light-emitting diodes in parallel.

10. The method of claim 7, wherein the electrical connections connect the plurality of light-emitting diodes using hybrid parallel and series circuitry.

11. The method of claim 7, wherein the biliary stent or catheter has an external diameter in a range of 12 Fr±4 Fr.

12. The method of claim 7, wherein the biliary stent or catheter has an internal diameter in a range of 1.3 mm±0.05 mm.

13. The method of claim 7, wherein the photosensitizes is a photosensitizer that, when excited, inhibits growth of bacteria.

14. The method of claim 7, wherein, the photosensitizer is a photosensitizer that, when activated, inhibits growth of cancer cells.
